(19) United States
(12) Patent Application Publication
(10) Pub. No.: US 2015/0086584 A1
(43) Pub. Date: Mar. 26, 2015

(54) MULTI-SPECIFIC BINDING AGENTS

(71) Applicant: University of Miami, Miami, FL (US)

(72) Inventors: Eli Gilboa, Coral Gables, FL (US); Randall Brennerman, Miami, FL (US); Brett Schrand, Miami, FL (US)

(73) Assignee: University of Miami, Miami, FL (US)

(21) Appl. No.: 14/386,903

(22) PCT Filed: Mar. 14, 2013
(86) PCT No.: PCT/US2013/031325

§ 371 Date: Sep. 22, 2014

Related U.S. Application Data
(60) Provisional application No. 61/614,034, filed on Mar. 22, 2012.

(51) Int. Cl. A61K 39/00; A61K 45/06

(52) U.S. Cl. CPC A61K 39/0011; A61K 39/0008; A61K 45/06; A61K 2039/70
USPC 424/192.1; 530/395

(57) ABSTRACT

Compositions comprising aptamers or other binding ligands provide immune cell regulatory signals and specificity to secreted cell molecules at in vivo foci of immune activity.

### FIELD OF THE INVENTION

Embodiments of the invention provide compositions and methods for highly selective targeting of therapeutic agents to molecules secreted into the extracellular spaces by cells or involved in a disease or disorder, including cells effecting an immune response. In particular, the therapeutic agents are specific for at least one molecule associated with an immune cell and at least one secreted cell molecule.

### BACKGROUND

In pluricellular organisms, cells communicate with each other via extracellular molecules such as nucleotides, lipids, short peptides, or proteins. These molecules are released extracellularly by cells and bind to receptors on other cells, thus inducing intracellular signaling and modification of the intracellular physiological state of the recipient cells. Extracellular signaling molecules are all fairly small, and are easily conveyed to the site of action; they are structurally very diverse. The classification and individual names of these mainly water-soluble mediators often reflect their first discovered action rather than their structure. Signaling via secreted signaling molecules can be paracrine (acting on neighboring cells), autocrine (acting on the cell that secretes the signaling molecule), endocrine (acting on cells that are remote from the secreting cell) or electrical (between two neurons or between a neuron and a target cell).

With respect to the immune system, extracellular signaling is one of the important factors in regulating an immune response. For example, induction of potent anti-pathogen or anti-tumor immunity requires not only antigenic stimulation but also co-stimulation mediated by ligands which interact with receptors on the surface of the immune cells, e.g. CD28, 4-1BB, OX40, etc.

### SUMMARY

This Summary is provided to present a summary of the invention to briefly indicate the nature and substance of the invention. It is submitted with the understanding that it will not be used to interpret or limit the scope or meaning of the claims.

Embodiments are directed to compositions comprising a multi specific agent having specificities for at least at two molecules, wherein a first domain is specific for a secreted cellular molecule and a second domain is specific for an immune cell or immune modulatory molecule. The domains of the agents comprise any molecule which can specifically bind to a target secreted cellular molecule or an immune cell modulatory antigen. In some embodiments, the immune modulatory molecule is an immune stimulatory molecule. In other embodiments, the immune modulatory molecule is an immune inhibitory molecule.

In some embodiments, the agent comprises aptamers, antibodies, antibody fragments, oligonucleotides, mimetics, peptides or small molecular weight (MW) compounds. which bind to secreted products, or combinations thereof.

In other embodiments, the multi-specific binding agent is specific for at least two immune cell modulatory molecule and at least one secreted cellular molecule.

In other embodiments, the multi-specific binding agent is specific for at least two secreted cellular molecules and at least one immune cell modulatory molecule.

In other embodiments, the secreted cellular molecule comprises molecules secreted by cells in tumor stroma comprising: CCL21, sialoproteins, cytokines, growth factors, tumor antigens, tumor associated antigens, peptides, or combinations thereof. Examples of growth factors, cytokines and angiogenic factors comprise, without limitation: Vascular endothelial growth factor (VEGF), tumor necrosis factors (TNF) transforming growth factors (TGF), colony stimulating factors (CSF), Fibroblast growth factors (FGF), epidermal growth factor (EGF), platelet-derived growth factor (PDGF), interferons (IFN), interleukins, endostatins, osteopontin (bone sialoprotein (BSP)), or fragments thereof.

In other embodiments, the secreted cellular molecule comprises secreted inflammatory molecules.

In yet other embodiments, the secreted cellular molecule comprises molecules secreted by cells in tissues or organs subjected to an autoimmune reaction.

In other embodiments, an immune cell stimulatory molecule comprises: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2, CD150 (SLAM), CD152 (CTLA-4), CD154, (CD40L), CD178 (FasL), CD209 (DC-SIGN), CD270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles or fragments thereof.

In other embodiments, an immune cell inhibitory molecule comprises: CTLA-4 (CD152), PD-1, or BTLA.

In other embodiments, one or more compositions are adminietered to patients in need of treatment, for example, cancer, autoimmunity, allergic reactions, neurological diseases, neuroinflammatory diseases, infections and the like.

Other aspects are described infra.

### BRIEF DESCRIPTION OF THE DRAWINGS

FIG. 1A (left panel)shows the results from experiments whereby mice were implanted with tumor, melanoma B16, and at day 3 or day 6 as indicated subjected to treatment which included either vaccination alone (GVAX) or vaccination and treatment with a bi-specific aptamer (conjugate) consisting of 4-1BB fused to VEGF. The conjugate enhanced tumor immunity. FIG. 1B (panel on the right) is a control w/o vaccination when treatment started at day 3 (with more material) which shows that the antitumor effect of the conjugate requires physical linkage, because 4-1BB and VEGF as a mixture had a minor effect by comparison.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, a “target cell” or “recipient cell” refers to an individual cell or cell which is desired to be, or has been, bound by the multi-specific agents. The term is also intended to include progeny of a single cell.

As used herein, the term “oligonucleotide,” includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like. The oligonucleotide may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register,” that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “gene” means the gene and all currently known variants thereof and any further variants which may be elucidated. For example, when referring to a particular antigen, such as, for example, VEGF, the term refers to all variants, mutants, alleles, species etc.

By the term “modulate,” it is meant that any of the mentioned activities, are, e.g., increased, enhanced, increased, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

As used herein, a “pharmaceutically acceptable” component/carrier etc is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

“Target molecule” or “secreted cellular molecule” includes any secreted macromolecule, by any cells present in a target microenvironment in vivo requiring a therapeutic intervention. For example, in the case of a tumor, this includes all cells, both normal and transformed or tumor cells, immune cells etc. In the case, for example, an inflammatory or autoimmune reaction, this includes both the normal cells, cells involved in the autoimmune reaction or any cell affected by the autoimmune reaction. Thus, any molecule secreted by any cell in the target microenvironment is a potential target, including proteins, sialoproteins, growth factors, cytokines, carbohydrate, enzyme, polysaccharide, glycoprotein, secreted receptors, antigen, antibody, growth factor; or it may be any small organic molecule including a hormone, substrate, metabolite, cofactor, inhibitor, drug, dye, nutrient, peptide; or it may be an inorganic molecule including a metal, metal ion, metal oxide, and metal complex; it may also be an entire organism including a bacterium, virus, and single-cell eukaryote such as a protozoon.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year.

### Compositions

In embodiments, a composition comprises a multi specific agent having specificities for at least at two molecules, wherein a first domain is specific for a secreted cellular molecule and a second domain is specific for an immune cell modulatory, e.g. stimulatory or inhibitory, molecule.

In embodiments, the agent comprises: aptamers, antibodies, antibody fragments, RGD-motif containing molecules, oligonucleotides, peptides, small molecular weight (MW) compounds which bind to secreted products (for example, metalloproteases), mimetics, or combinations thereof. For example, the agent can be engineered to comprise an aptamer and an antibody fragment, or an oligonucleotide and an integrin. Mimetics can also be employed. As used herein, a “mimetic” would represent the molecule which binds to the secreted molecule. For example, for a growth factor, e.g. VEGF, the molecule which binds to the secreted VEGF can be one which mimics the VEGF receptor. The types of mimetics used are limited only by the imagination of the user. Discussion of the invention will use the term “aptamers” for illustrative and descriptive purposes. However, it is not meant to construe or limit the invention in any way. The invention is meant to cover all and any means of agents which specifically bind to a secreted cellular molecule. So, for example, a small molecular weight compound would be one that would bind to a metalloprotease, a growth factor, cytokine etc.

Aptamers are oligonucleotide or peptide molecules that bind to a specific target molecule. Aptamers are usually created by selecting them from a large random sequence pool, but natural aptamers can also exist in riboswitches. More specifically, aptamers can be classified as: DNA or RNA aptamers and comprise strands of oligonucleotides. Peptide aptamers comprise short variable peptide domains, attached at both ends to a protein scaffold.

As used herein, the term “aptamer” refers to bi-specific or multi-specific molecules. For example, the aptamer can bind to two or more target molecules and two or more immune cell modulatory molecules which includes, inhibitory, stimulatory, co-stimulatory or co-inhibitory antigens. The combinations of specificities can be determined by the user and as such, provides for unlimited combinations of specificities.

In some embodiments, the multi-specific binding agent is specific for at least two immune cell stimulatory molecule and at least one secreted cellular molecule.

In other embodiments, the multi-specific binding agent is specific for at least two secreted cellular molecules and at least one immune cell modulatory molecule.

In some embodiments, the multi-specific binding agent is a bi-specific aptamer.

Since development and progression of tumors is not only dependent on cancer cells themselves but also on the active contribution of the stromal cells, e.g. by secreting growth supporting factors, enzymes degrading the extracellular matrix or angiogenic factors, the tumor stroma serve as a target for immune intervention. In some embodiments, the multi-specific agent is targeted in vivo to diseased or abnormal tissues, by targeting secreted cellular products into the extracellular spaces. The disease or abnormal areas comprise for example, tumors, cells and tissues involved in an auto-immune reaction, areas of inflammation, wounds, burns and the like. In one embodiment, the multi-specific binding agent is specific for secreted cellular molecules comprising tumor molecules secreted into a tumor stroma from any cell, e.g. including normal cells. In another embodiment, the multi-specific binding agent is specific for secreted cellular molecules comprising secreted inflammatory molecules. In another embodiment, the multi-specific binding agent is specific for secreted cellular molecules comprising secreted by cells in tissues or organs subjected to an autoimmune reaction.

Tumor cells influence the immune system by inducing changes in their microenvironment. Administering to patients compositions which target products upregulated in the tumor stroma inhibit tumor growth. Embodiments of the invention comprise a bispecific or multi-specific costimulatory agent which binds to molecules secreted into the tumor stroma, by either tumor cells or stromal cells which make up the tumor stroma, also delivers an immune cell co-stimulatory signal, activating immune cells specific for a desired stromal secreted product. For example, lymphoid tissues are composed of a highly organized network of stromal cells that bring foreign antigens and immune cells into close contact to initiate adaptive immune responses. The formation of lymphoid tissues is coordinated by lymphoid tissue-induced (LTi) cells, which promote the localized expression of chemokines. LTi cells stimulate lymphoid stromal cells, such as fibroblast-like reticular cells (FRCs), to release chemokines that recruit antigen-presenting dendritic cells and lymphocytes into the lymphoid tissue. 6 FRCs secrete CCL19 and CCL21, stimulating the recruitment of CCR7⁺ cells, such as naive and memory T cells, mature dendritic cells, and LTi cells.

Though it seems counterproductive for tumors to create a peripheral environment that is similar to immune tissue, lymphoid tissue can promote immune tolerance. For example, lymph node stromal cells present peripheral tissue antigens to circulating T cells to induce peripheral tolerance. Thus, it is plausible that the newly created lymphoid-like stromal structures aid tumor cells in immune system evasion and consequently, enhance tumor growth. In such embodiments, the multi-specific binding agent comprises specificity ofr a secreted molecule which inhibits the immune repsonse and an immune stimulatory or co-stimulatory domain for modulating the immune response to overcome the tolerogenic mechanisms.

There are several potential mechanisms that CCL21-secreting tumor cells may utilize for inducing immune tolerance (Shields, J. D. et al. (2010) Science 328:749). CCL21-secreting tumors recruited more CD11b⁺CD11c⁻F4/80-Grl^{high} myeloid-derived suppressor cells (MDSCs) and regulatory T (Treg) cells. Additionally, two factors that facilitate tumor growth, indoleamine 2,3-dioxygenase (IDO) and complement receptor 1-related gene/protein y (Crry), were expressed by CCL21-secreting tumors, but were not present in CCL21^{low} tumors. CCL21^{low} tumors also recruited more mature and cytotoxic T cells specific for melanoma antigen, and had elevated levels of IFN-gamma, IL-2, and IL-4, cytokines associated with anti-tumor immunity and cytotoxic T cell responses. Thus, the multi-specific binding agents embodied herein, provide new avenues for possible cancer treatments, including inhibition of CCL21 secretion by tumor cells or suppression of LTi cell function.

To target the monospecific (e.g. directed to the same antigen or directed to the same antigen but to different epitopes), bispecific or multi-specific immune comodulatory binding agents embodied by the invention to the tumors in vivo, a molecule specific for an immune cell modulatory receptor is conjugated, linked, fused and the like, to a second molecule which binds to molecules secreted by tumor cells or any of the normal cell constituents of the tumor stroma, referred to herein as “stromal cell(s).” Normal cell constituents of the tumor stroma comprise for example, cells such as macrophages, dendritic cells, endothelial cells, fibroblasts and the like. The bispecific or multi-specific agent specific for an immune cell immunomodulatory molecule can be for example, an aptamer, antibody, peptide and the like. The ligand or molecule which binds to secreted stromal cell molecules endothelial specific molecules such as, for example, vascular endothelial growth factor (VEGF), sialoproteins(e.g. osteopontin), CC chemokines (e.g. CCL21) and the like. One of skill in the art will appreciate that any other secreted antigen or protein associated with vascular or other tumor-associated stromal cells can be a target for the immunogenic compositions, including those that are presently known and those yet to be identified.

In a preferred embodiment, a bispecific or multi-specific binding agent comprises a domain or ligand which is specific for an immune cell co-stimulatory molecule, for example, a 4-1BB and second domain specific for products expressed on the surface of tumor cells, tumor stroma cells, and normal cells which make up the tumor stroma. An example of targeting ligand would be, for example, metalloproteases, chemokines, VEGF, osteopontin and the like. Examples of metalloproteases, include ADAMTSs (A Disintegrin and Metalloprotease with ThromboSpondin type 1 motif), BMP-1 (Bone morphogenetic protein 1 also known as procollagen Cproteinase (PCP)), MMPs (Matrix Metalloproteinases) and Pappalysins, In other embodiments, the bispecific or multi-specific costimulatory agent comprises combinations of one or more domains binding to co-stimulatory molecules and one or more domains which bind to cells in the tumor stroma. For example, 4-1BB is a major costimulatory receptor expressed on CD8⁺ T cells VEGF is a molecule secreted by cells of the tumor stroma.

In a preferred embodiment, the compositions of the present invention are targeted to immune cell modulatory molecules, for example, 4-1BB, CD27 (CD27 ligand is CD70), HVEM, LTβ receptors or ligands thereof.

In embodiments, the multi-specific binding agent is specific for at least one tumor secreted molecule comprising: growth factors, tumor antigens, cytokines, angiogenic factors, adhesion molecules, sialoproteins (e.g. osteopontin), integrins, carbohydrate structures, cell surface molecules, intra-cellular molecules, polynucleotides, oligonucleotides, proteins, peptides or receptors thereof.

Since tumor development and growth depends on the ability of tumor cells to evade the host's immune system, tumor cells employ strategies to impede anti-tumor immune responses, including secretion of immunosuppressive factors and activation of negative regulatory pathways. For example, invasive tumor cells secrete CCL21. In embodiments, the multi-specific agents target such secreted molecules.

In some embodiments, secreted molecules such as, growth factors, cytokines and angiogenic factors comprise: Vascular endothelial growth factor (VEGF), tumor necrosis factors (TNF) transforming growth factors (TGF), colony stimulating factors (CSF), Fibroblast growth factors (FGF), epidermal growth factor (EGF), platelet-derived growth factor (PDGF), interferons (IFN), interleukins, endostatins, osteopontin (bone sialoprotein (BSP)),chemokines (e.g. CCL21) or fragments thereof.

Immune Cell Modulation: In other preferred embodiments, the multi-binding agent comprises specificity for an immune stimulatory molecule and a secreted cellular molecule is specific for stimulatory and/or co-stimulatory molecules involved in immune reactions.

Co-stimulation of immune cells is mediated by ligands which interact with receptors on the surface of the immune cells, e.g. CD28, 4-1BB, OX40, etc. Tumor cells do not express costimulatory ligands and hence presentation of tumor antigens by the tumor cells does not potentiate the naturally occurring or a vaccine-induced antitumor immune response. Studies in mice and cancer patients have shown that tumors are recognized by the immune system and can elicit an immune response which controls tumor progression. Yet, this naturally occurring tumor-induced immune response is weak and has a limited impact in delaying, but not reversing, tumor progression. A main reason why tumors are not “immunogenic” is that they don't express costimulatory ligands to promote the survival and expansion of the tumor-infiltrating T cells.

In embodiments, the multi-binding agent comprises an immune cell modulatory molecule comprises: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2, CD150 (SLAM), CD152 (CTLA-4), CD154, (CD40L), CD178 (FasL), CD209 (DC-SIGN), CD270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles or fragments thereof.

To target the multi-specific binding agent to the tumors and tumor stroma in vivo the multi-specific binding agent was engineered for specificity for secreted cell molecules, such as, for example, VEGF, and an immune cell molecule associated with immune cell reactions, for example, 4-1BB.

In some embodiments the multi-specific binding agent is a bi-specific aptamer with specificity for VEGF-4-1BB.

In other embodiments the multi-specific binding agent is a bi-specific aptamer with specificity for osteopontin-4-1-BB.

In another preferred embodiments, the multi-specific binding agent may comprise aptamers specific for one or more immune cell stimulatory or inhibitory molecules and one or more tumor antigens.

In yet another preferred embodiment, the multi-specific binding agents bind to two cell secreted molecules, and an immune cell to effect a localized immune response. For example, if the abnormal cell is a tumor cell, the aptamer binds to a desired antigen secreted into the tumor stroma and the immune cell thus providing a co-stimulatory signal. The advantage is that an immune response is localized.

The term “abnormal cell” refers to any cell which is not physiologically normal, for example, a tumor cell; a cell infected with an organism; transformed cell; a cell whereby the surface molecules are affected, such as, glycosylation or decrease in receptors etc; a cell which induces an autoimmune response; a cell which produces a mutant polynucleotide etc. Any cell which does not resemble a physiological or genetically normal cell would be considered an abnormal cell.

Immune System: Immune systems are classified into two general systems, the “innate” or “primary” immune system and the “acquired/adaptive” or “secondary” immune system. It is thought that the innate immune system initially keeps the infection under control, allowing time for the adaptive immune system to develop an appropriate response. Studies have suggested that the various components of the innate immune system trigger and augment the components of the adaptive immune system, including antigen-specific B and T lymphocytes (Kos, Immunol. Res. 1998, 17:303; Romagnani, Immunol. Today. 1992, 13: 379; Banchereau and Steinman, Nature. 1988, 392:245).

A “primary immune response” refers to an innate immune response that is not affected by prior contact with the antigen. The main protective mechanisms of primary immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells (destroy microbes and certain tumor cells, and attack certain virus infected cells), and the inflammatory response (mobilizes leukocytes such as macrophages and dendritic cells to phagocytose invaders).

Some cells of the innate immune system, including macrophages and dendritic cells (DC), function as part of the adaptive immune system as well by taking up foreign antigens through pattern recognition receptors, combining peptide fragments of these antigens with major histocompatibility complex (MHC) class I and class II molecules, and stimulating naive CD8⁺ and CD4⁺ T cells respectively (Banchereau and Steinman, supra; Holmskov et al., Immunol. Today. 1994, 15:67; Ulevitch and Tobias Annu. Rev. Immunol. 1995, 13:437). Professional antigen-presenting cells (APCs) communicate with these T cells, leading to the differentiation of naïve CD4⁺ T cells into T-helper 1 (Th1) or T-helper 2 (Th2) lymphocytes that mediate cellular and humoral immunity, respectively (Trinchieri Annu. Rev. Immunol. 1995, 13:251; Howard and O'Garra, Immunol. Today. 1992, 13:198; Abbas et al., Nature. 1996, 383:787; Okamura et al., Adv. Immunol. 1998, 70:281; Mosmann and Sad, Immunol. Today. 1996, 17:138; O'Garra et al., Immunity. 1998, 8:275).

A “secondary immune response” or “adaptive immune response” may be active or passive, and may be humoral (antibody based) or cellular that is established during the life of an animal, is specific for an inducing antigen, and is marked by an enhanced immune response on repeated encounters with said antigen. A key feature of the T lymphocytes of the adaptive immune system is their ability to detect minute concentrations of pathogen-derived peptides presented by MHC molecules on the cell surface. Upon activation, naïve CD4 T cells differentiate into one of at least two cell types, Thl cells and Th2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response, and reciprocally, activation of the Th2 type cytokine response can suppress the Th1 type response.

In adaptive immunity, adaptive T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

The phrase “T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

“Cells of the immune system” or “immune cells”, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, tumor-infiltrating (TIL) cells, gene modified immune cells including hybridomas, drug modified immune cells, antigen presenting cells and derivatives, precursors or progenitors of the above cell types.

Thus, in embodiments, the multi-specific agent modulates immune cells and can either stimulate, such as for example, an anti-tumor response, or inhibit, such as for example, autoimmunity, inflammatory or allergic responses.

“Immune effector cells” refers to cells, and subsets thereof, e.g. Treg, Th1, Th2, capable of binding an antigen and which mediate an immune response selective for the antigen. These cells include, but are not limited to, T cells (T lymphocytes), B cells (B lymphocytes), antigen presenting cells, such as for example dendritic cells, monocytes, macrophages; myeloid suppressor cells, natural killer (NK) cells and cytotoxic T lymphocytes (CTLs), for example CTL lines, CTL clones, and CTLs from tumor, inflammatory, or other infiltrates.

A “T regulatory cell” or “Treg cell” or “Tr cell” refers to a cell that can inhibit a T cell response. Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by MSC activity. Several Treg subsets have been identified that have the ability to inhibit autoimmune and chronic inflammatory responses and to maintain immune tolerance in tumor-bearing hosts. These subsets include interleukin 10—(IL-10-)secreting T regulatory type 1 (Tr1) cells, transforming growth factor-β—(TGF-β-) secreting T helper type 3 (Th3) cells, and “natural” CD4⁺/CD25⁺ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

The term “myeloid suppressor cell (MSC)” refers to a cell that is of hematopoietic lineage and expresses Gr-1 and CD11b; MSCs are also referred to as immature myeloid cells and were recently renamed to myeloid-derived suppressor cells (MDSCs). MSCs may also express CD115 and/or F4/80 (see Li et al., Cancer Res. 2004, 64:1130-1139). MSCs may also express CD31, c-kit, vascular endothelial growth factor (VEGF)-receptor, or CD40 (Bronte et al., Blood. 2000, 96:3838-3846). MSCs may further differentiate into several cell types, including macrophages, neutrophils, dendritic cells, Langerhan's cells, monocytes or granulocytes. MSCs may be found naturally in normal adult bone marrow of human and animals or in sites of normal hematopoiesis, such as the spleen in newborn mice. Upon distress due to graft-versus-host disease (GVHD), cyclophosphamide injection, or γ-irradiation, for example, MSCs may be found in the adult spleen. MSCs can suppress the immunological response of T cells, induce T regulatory cells, and produce T cell tolerance. Morphologically, MSCs usually have large nuclei and a high nucleus-to-cytoplasm ratio. MSCs can secrete TFG-β and IL-10 and produce nitric oxide (NO) in the presence of IFN-γ or activated T cells. MSCs may form dendriform cells; however, MSCs are distinct from dendritic cells (DCs) in that DCs are smaller and express CD11c; MSCs do not express CD11c. T cell inactivation by MSCs in vitro can be mediated through several mechanisms: IFN-γ-dependent nitric oxide production (Kusmartsev et al. J Immunol. 2000, 165: 779-785); Th2-mediated-IL-4/IL-13-dependent arginase 1 synthesis (Bronte et al. J Immunol. 2003, 170: 270-278); loss of CD3ζ signaling in T cells (Rodriguez et al. J Immunol. 2003, 171: 1232-1239); and suppression of the T cell response through reactive oxygen species (Bronte et al. J Immunol. 2003, 170: 270-278; Bronte et al. Trends Immunol. 2003, 24: 302-306; Kusmartsev et al. J Immunol. 2004, 172: 989-999; Schmielau and Finn, Cancer Res. 2001, 61: 4756-4760).

Numerous costimulatory molecules have been identified playing a role in the initiation of immune responses by T and B lymphocytes. Signals provided through CD28-B7 interactions are essential for initial naive T cell activation leading to increased IL-2 production and IL-2Rα (CD25) expression. NKG2D binds to the MHC-related proteins MIC and Rae-1 and induces IL-2 production and proliferation. In other cell types, such as B cells, activation requires CD4O-CD40L interactions for proper antibody response: promoting survival, cytokine receptor expression, and inducing antibody class switch. In addition to the costimulatory pathways that are important in naive lymphocyte activation, other costimulatory molecules play a role in effector/memory lymphocyte activation.

The costimulatory receptors ICOS, OX-40, 4-1BB, and CD27 bind to their ligands B7h, OX-40L, 4-1BBL, and CD70, respectively, to enhance the activation, survival, and cytokine secretion of effector/memory, but not naive T and B cells. These costimulatory receptors and their ligands are not constitutively expressed but are induced on differentiated T cells, and their ligands are not restricted to APCs. T cell activation generally incorporates a self-limiting mechanism, such as inhibitory costimulators, to regulate T cell tolerance and attenuate the immune response. The expanding set of inhibitory costimulators currently includes CTLA-4 (CD152), PD-1, and BTLA. While expression of these molecules is induced following T cell activation, they are absent on nave T cells. Lastly, B7-H3 is a new costimulatory ligand originally described to induce T cell proliferation and IFN-γ production through an as of yet unidentified receptor.

In preferred embodiments, the immune cell co-stimulatory induce an immune response. Examples of immune cell co-stimulatory molecules comprise: 4-1BB (CD137), OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD154, (CD40L), CD178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

Examples of immune cells comprise T cells (T lymphocytes), B cells (B lymphocytes), antigen presenting cells, dendritic cells, monocytes, macrophages, myeloid suppressor cells, natural killer (NK) cells, NKT cells, NKT suppressor cells, T regulatory cells (Tregs), T suppressor cells, cytotoxic T lymphocytes (CTLs), CTL lines, CTL clones, CTLs from tumor, inflammatory, or other infiltrates and subsets thereof.

Natural killer T (NKT) cells are a heterogeneous group of T cells that share properties of both T cells and natural killer (NK) cells. Many of these cells recognize the nonpolymorphic CD1d molecule, an antigen-presenting molecule that binds self- and foreign lipids and glycolipids. NKT cells are a subset of T cells that co-express an αβ T cell receptor (TCR), but also express a variety of molecular markers that are typically associated with NK cells, such as NK1.1. They differ from conventional áâ T cells in that their TCRs are far more limited in diversity and in that they recognize lipids and glycolipids presented by CD1d molecules, a member of the CD1 family of antigen presenting molecules, rather than peptide-MHC complexes. NKT cells include both NK1.1⁺ and NK1.1−, as well as CD4⁺, CD4⁻, CD8⁺ and CD8⁻ cells. Natural Killer T cells share other features with NK cells as well, such as CD 16 and CD56 expression and granzyme production. NKT cells are classified into type I (invariant) and type II (non-invariant) cells in mice and humans. The best known subset of CD1d-dependent NKT cells expresses an invariant T cell receptor a (TCR-α) chain. These are referred to as type I or invariant NKT cells (iNKT) cells.

Originally called suppressor T cells (Ts cells), the most promising recent candidates have been termed regulatory T cells (Treg cells). Treg cells are a specialized subpopulation of T cells that act to suppress activation of the immune system and thereby maintain immune system homeostasis and tolerance to self-antigens. Regulatory T cells come in many forms, including those that express the CD8 transmembrane glycoprotein (CD8+ T cells), those that express CD4, CD25 and Foxp3 (CD4+CD25+ regulatory T cells or “Tregs”) and other T cell types that have suppressive function. These cells are involved in closing down immune responses after they have successfully tackled invading organisms, and also in keeping in check immune responses that may potentially attack one's own tissues (autoimmunity).

CD4⁺ Foxp3⁺ regulatory T cells have been referred to as “naturally-occurring” regulatory T cells to distinguish them from “suppressor” T cell populations that are generated in vitro. Additional suppressive T cell populations include Tr1, CD8⁺CD28⁻, and Qa-1 restricted T cells.

In preferred embodiments, an aptamer binds to one or more immune cell type molecules involved with modulating an immune response and one or more antigens secreted by the target cells, such as for example, one or more tumor cell antigens secreted into the tumor stroma.

In another preferred embodiment, a multi-specific binding agent specifically binds to one or more co-stimulatory immune cell molecules and one or more antigens secreted by cells involved with or associated with an immune reaction in vivo. As used herein, “immune reaction” is meant to include any cells or cell products involved in an immune response, including, the target cells or target cell products to which the immune reaction has been generated against. For example, in autoimmune reactions, the target cells could be islet cells, as in the case of diabetes, and the molecules could be any molecule secreted by the islet cells.

The aptamers described herein, can be generated to be specific for antigens secreted by tumor cells, antigens secreted by cells that are involved in autoimmune reactions, antigens secreted by cells involved in inflammations, antigens secreted by infected cells and the like.

The aptamer specificity can be tailored to bind to stimulatory or co-stimulatory molecules, including any immune cell molecule that may be needed to initiate a suppressive immune reaction, as in the case, for example, of an autoimmune reaction.

Lack of costimulation: Melanoma tumor cells are immunogenic; theoretically, they should cause an immune response but they do not stimulate an effective anti-tumor immune response in vivo. Melanoma tumors may be capable of delivering antigen-specific signals to T cells, but do not deliver the co-stimulatory signals necessary for full activation of T cells because of the lack of B7 expression on their surface. T cell activation requires two distinct signaling events. The first signal originates from the binding of the TCR to its antigen-MHC ligand, and provides the specificity of the interaction. The second signal is either provided by soluble factors such as IL-2 or the interaction of cell-surface molecules on the T cell with their ligands on APCs. This second signal is thought to provide the necessary costimulation to the TCR-mediated signaling event. Binding of the TCR with peptide-MHC complexes in the absence of costimulation can result in T cell inactivation or anergy, which is associated with a block in the IL-2 gene transcription. For example, expression of B7 on the surface of a cell is the costimulatory signal necessary to allow for the cytolytic CD8⁺ T cell attack on the tumor. The costimulation results from an interaction of the CD28 molecule on the T cell surface with its ligand, B7, on the surface of an antigen-presenting cell (APC). B7 display renders tumor cells capable of effective antigen presentation, leading to their eventual eradication.

In preferred embodiments, enhancing or inducing the immunogenicity of a tumor cell in vivo comprises administering to a patient a composition comprising a bi- or multi-specific aptamer which binds to secreted tumor antigens and immune cell modulatory molecules. Thus, the aptamer composition modulates the functions of the cells, for example, proliferation of a lymphocyte wherein that lymphocyte had been previously suppressed or attenuated.

The cell can be any type of one or more immune cells. In some preferred embodiments, the immune cell is a lymphocyte. These reagents or compositions involved or associated with modulating immunity, such as costimulation (i.e., CTLA-4, 4-1BB, PD-1, etc.) serve as important adjunct to, or replace altogether, new and powerful, often complex, vaccination protocols currently under development.

In another preferred embodiment, the bi- or multi-specific aptamer compositions target cells involved in rendering the immune system tolerant to a particular antigen or antigens.

“Tolerance” refers to the anergy (non-responsiveness) of immune cells, e.g. T cells, when presented with an antigen. T cell tolerance prevents a T cell response even in the presence of an antigen that existing memory T cells recognize.

In another preferred embodiment, the aptamers can be used in to treating any disease wherein immunogenicity of a target is desired, for example, viral diseases.

In other embodiments, the multi-specific agents optionally comprise a cargo moiety to deliver a further therapeutic agent to the target microenvironment. Examples include, a radioactive agent, a label for imaging or diagnostic purposes, toxins, anti-inflammatory molecules, small molecules and the like.

In preferred embodiments, the oligonucleotides can be tailored to individual therapy, for example, these oligonucleotides can be sequence specific for allelic variants in individuals, the up-regulation in immunogenicity of a target can be manipulated in varying degrees, such as for example, 10%, 20%, 40%, 100% expression relative to the control. That is, in some patients it may be effective to increase immunogenicity by 10% versus 80% in another patient.

Immunogenicity of a target can be monitored by various techniques known in the art such as, immuno assays, blotting, and the like.

Aptamer composition: By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

The aptamer may be linked to one or more other aptamers with similar or varying specificities by a linker. A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jaschke et al., Tetrahedron Lett. 34:301, 1993; Ono et al., Biochemistry 30:9914, 1991).

The invention may be used against protein coding gene products as well as nonprotein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like.

In another preferred embodiment, the nucleobases in the aptamers may be modified to provided higher specificity and affinity for a target. For example nucleobases may be substituted with LNA monomers, which can be in contiguous stretches or in different positions. The modified molecules, preferably have a higher association constant (Ka) for the target sequences than the complementary sequence. Binding of the modified or non-modified molecules to target sequences can be determined in vitro under a variety of stringency conditions using hybridization assays.

Certain preferred aptamer oligonucleotides of this invention are chimeric oligonucleotides. “Chimeric oligonucleotides” or “chimeras,” in the context of this invention, are oligonucleotides which contain two or more chemically distinct regions, each made up of at least one nucleotide. These oligonucleotides typically contain at least one region of modified nucleotides that confers one or more beneficial properties, such as, for example, increased nuclease resistance, increased binding affinity for the target molecule. Consequently, comparable results can often be obtained with shorter oligonucleotides when chimeric oligonucleotides are used, compared to phosphorothioate deoxyoligonucleotides hybridizing to the same target region.

In one preferred embodiment, a chimeric oligonucleotide comprises at least one region modified to increase target binding affinity. Affinity of an oligonucleotide for its target is routinely determined by measuring the Tₘ of an oligonucleotide/target pair, which is the temperature at which the oligonucleotide and target dissociate; dissociation is detected spectrophotometrically. The higher the Tₘ, the greater the affinity of the oligonucleotide for the target.

In another preferred embodiment, the region of the oligonucleotide which is modified comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrymidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target. The effect of such increased affinity is to greatly enhance RNA interference (RNAi) oligonucleotide inhibition of gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ [known as a methylene(methylimino) or MMI backbone], CH₂—O—N (CH₃)—CH₂, CH₂—N (CH₃)—N (CH₃)—CH₂ and 0—N (CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497).

Oligonucleotides may also comprise one or more substituted sugar moieties.

Preferred oligonucleotides comprise one of the following at the 2′ position: OH, SH, SCH₃, F, OCN, OCH₃ OCH₃, OCH₃ O(CH₂)ₙ CH₃, O(CH₂)ₙ NH₂ or O(CH₂)ₙ CH₃ where n is from 1 to about 10; C₁ to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂ CH₂ OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety

(Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, the nucleic acid molecule of the present invention is conjugated with another moiety including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

In accordance with the invention, use of modifications such as the use of LNA monomers to enhance the potency, specificity and duration of action and broaden the routes of administration of oligonucleotides comprised of current chemistries such as MOE, ANA, FANA, PS etc (ref: Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 5 and 25 nucleotides, more preferably between about 12 and 20 nucleotides.

Preferred modified oligonucleotide backbones comprise, but not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

In another preferred embodiment of the invention the oligonucleotides with phosphorothioate backbones and oligonucleosides with heteroatom backbones, and in particular —CH₂—NH—O—CH₂—, —CH₂—N (CH₃)—O—CH₂— known as a methylene (methylimino) or MMI backbone, —CH₂—O—N (CH₃)—CH₂—, —CH₂N(CH₃)—N(CH₃) CH₂— and —O—N(CH₃)—CH₂—CH₂— wherein the native phosphodiester backbone is represented as —O—P—O—CH₂— of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O alkyl-O-alkyl, wherein the alkyl, alkenyl and alkynyl may be substituted or unsubstituted C to CO alkyl or C₂ to CO alkenyl and alkynyl. Particularly preferred are O (CH₂)ₙ OₘCH₃, O(CH₂)ₙ, OCH₃, O(CH₂)ₙNH₂, O(CH₂)nCH₃, O(CH₂)ₙONH₂, and O(CH₂ₙON(CH₂)nCH₃)₂ where n and m can be from 1 to about 10. Other preferred oligonucleotides comprise one of the following at the 2′ position: C to CO, (lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification comprises 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification comprises 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N (CH₂)₂.

Other preferred modifications comprise 2′-methoxy (2′-O CH₃), 2′-aminopropoxy (2′-O CH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Generation of Aptamers: Aptamers are high affinity single-stranded nucleic acid ligands which can be isolated from combinatorial libraries through an iterative process of in vitro selection known as SELEX™ (Systemic Evolution of Ligands by EXponential enrichment). Aptamers exhibit specificity and avidity comparable to or exceeding that of antibodies, and can be generated against most targets. Unlike antibodies, aptamers, can be synthesized in a chemical process and hence offer significant advantages in terms of reduced production cost and much simpler regulatory approval process. Also, aptamers are not expected to exhibit significant immunogenicity in vivo.

In preferred embodiments, at least one aptamer is linked to at least one other aptamer which is specific for a desired cell antigen and a stimulatory and/or co-stimulatory immune cell target molecule. In other embodiments, a plurality of aptamers can be directed to different target molecules and stimulatory and/or co-stimulatory molecules. The various permutations and combinations for combining aptamers is limited only by the imagination of the user.

Methods of the present disclosure do not require a priori knowledge of the nucleotide sequence of every possible gene variant (including mRNA splice variants) targeted. Aptamers specific for a given biomolecule can be identified using techniques known in the art. See, e.g., Toole et al. (1992) PCT Publication No. WO 92/14843; Tuerk and Gold (1991) PCT Publication No. WO 91/19813; Weintraub and Hutchinson (1992) PCT Publication No. 92/05285; and Ellington and Szostak, Nature 346:818 (1990). Briefly, these techniques typically involve the complexation of the molecular target with a random mixture of oligonucleotides. The aptamer-molecular target complex is separated from the uncomplexed oligonucleotides. The aptamer is recovered from the separated complex and amplified. This cycle is repeated to identify those aptamer sequences with the highest affinity for the molecular target.

In yet another aspect, aptamers that selectively bind to variants of target gene expression products can be identified, e.g. new tumor antigens, or other types of desired antigen or stimulatory molecule targets. A “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Sequence similarity searches can be performed manually or by using several available computer programs known to those skilled in the art. Preferably, Blast and Smith- Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

In accordance with the invention, paralogs can be identified for designing the appropriate aptamers. Paralogs are genes within a species that occur due to gene duplication, but have evolved new functions, and are also referred to as isotypes.

### Pharmaceutical Compositions

The invention also includes pharmaceutical compositions containing nucleic acid conjugates. In some embodiments, the compositions are suitable for internal use and include an effective amount of a pharmacologically active conjugate of the invention, alone or in combination, with one or more pharmaceutically acceptable carriers. The conjugates are especially useful in that they have very low, if any toxicity.

The patient having a pathology, e.g. the patient treated by the methods of this invention can be a mammal, or more particularly, a human. In practice, the aptamers, are administered in amounts which will be sufficient to exert their desired biological activity.

The pharmaceutical compositions of the invention may contain, for example, more than one aptamer specificity. In some examples, a pharmaceutical composition of the invention, containing one or more compounds of the invention, is administered in combination with another useful composition such as an anti-inflammatory agent, an immunostimulator, a chemotherapeutic agent, an antiviral agent, or the like. Furthermore, the compositions of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

Combination therapy (or “co-therapy”) includes the administration of an aptamer composition and at least a second agent as part of a specific treatment regimen intended to provide the beneficial effect from the co-action of these therapeutic agents. The beneficial effect of the combination includes, but is not limited to, pharmacokinetic or pharmacodynamic coactions resulting from the combination of therapeutic agents. Administration of these therapeutic agents in combination typically is carried out over a defined time period (usually minutes, hours, days or weeks depending upon the combination selected).

Combination therapy may, but generally is not, intended to encompass the administration of two or more of these therapeutic agents as part of separate monotherapy regimens that incidentally and arbitrarily result in the combinations of the present invention. Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

The multi-specific binding agents can be formulated according to known methods to prepare pharmaceutically useful compositions, whereby the compound is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™M. (ICI Americas Inc., Bridgewater, N.J.), PLURONICS™ (BASF Corporation, Mount Olive, N.J.) or PEG.

The formulations to be used for in vivo administration must be sterile and pyrogen free. This is readily accomplished by filtration through sterile filtration membranes, prior to or following lyophilization and reconstitution.

The route of administration is in accord with known methods, e.g.

injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

Formulations for oral administration in the present invention may be presented as: discrete units such as capsules, cachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavoring agents such as peppermint, oil of wintergreen, cherry flavoring and the like can also be used. It may be desirable to add a coloring agent to make the dosage form readily identifiable. Tablets may also be coated by methods well known in the art.

A tablet may be made by compression or molding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavored base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

Non-limiting examples of methods and compositions disclosed herein are as follows:
1. A composition comprising: a multi specific agent having specificities for at least at two molecules, wherein a first domain is specific for a secreted cellular molecule and a second domain is specific for an immune cell modulatory molecule.
2. The composition of embodiment 1, wherein the agent comprises: aptamers, antibodies, antibody fragments, oligonucleotides, mimetics, peptides or small molecular weight (MW) compounds which bind to secreted products.

3. The composition of embodiment 2, wherein the small molecular weight compounds bind to secreted cellular molecules.
4. The composition of embodiment 3, wherein the small molecular weight compounds bind to metalloproteases.
5. The composition of embodiment 1, wherein the multi-specific binding agent is specific for at least two immune cell modulatory molecule and at least one secreted cellular molecule.
6. The composition of embodiment 1, wherein the multi-specific binding agent is specific for at least two secreted cellular molecules and at least one immune cell modulatory molecule.
7. The composition of embodiment 1, wherein the immune modulatory molecule is an immune stimulatory molecule.
8. The composition of embodiment 1, wherein the immune modulatory molecule is an immune inhibitory molecule.
9. The composition of embodiment 1, wherein the multi-specific binding ligand is a bi-specific aptamer.
10. The composition of embodiment 1, wherein the secreted cellular molecule comprises molecules secreted by cells in tumor stroma comprising: CCL21, sialoproteins, cytokines, growth factors, tumor antigens, tumor associated antigens, peptides, or combinations thereof.
11. The composition of embodiment 10, wherein growth factors, cytokines and angiogenic factors comprise: Vascular endothelial growth factor (VEGF), tumor necrosis factors (TNF) transforming growth factors (TGF), colony stimulating factors (CSF), Fibroblast growth factors (FGF), epidermal growth factor (EGF), platelet-derived growth factor (PDGF), interferons (IFN), interleukins, endostatins, osteopontin (bone sialoprotein (BSP)), or fragments thereof.
12. The composition of embodiment 1, wherein the secreted cellular molecule comprises secreted inflammatory molecules.
13. The composition of embodiment 1, wherein the secreted cellular molecule comprises molecules secreted by cells in tissues or organs subjected to an autoimmune reaction.
14. The composition of embodiment 7, wherein an immune cell stimulatory molecule comprises: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2, CD150 (SLAM), CD152 (CTLA-4), CD154, (CD40L), CD178 (FasL), CD209 (DC-SIGN), CD270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, T Cell receptor αβ (TCRαβ), T Cell receptor γδ (TCRγδ), T cell receptor ζ (TCRζ), TGFβRII, TNF receptor, Cd11c, CD 1-339, B7, Foxp3, mannose receptor, or DEC205, variants, mutants, species variants, ligands, alleles or fragments thereof.
15. The composition of embodiment 8, wherein an immune cell inhibitory molecule comprises: CTLA-4 (CD152), PD-1, or BTLA.
16. The composition of embodiment 1, optionally comprising one or more cargo moieties comprising: a chemotherapeutic agent, toxin, radioactive agent, enzyme, small molecule, organic compound, inorganic compound, or combinations thereof.
17. A method of treating cancer comprising administering to a patient a therapeutically effective amount of a composition of embodiment 1.
18. A method of treating autoimmune related diseases or disorders comprising administering to a patient a therapeutically effective amount of a composition of embodiment 1.

### EXAMPLES

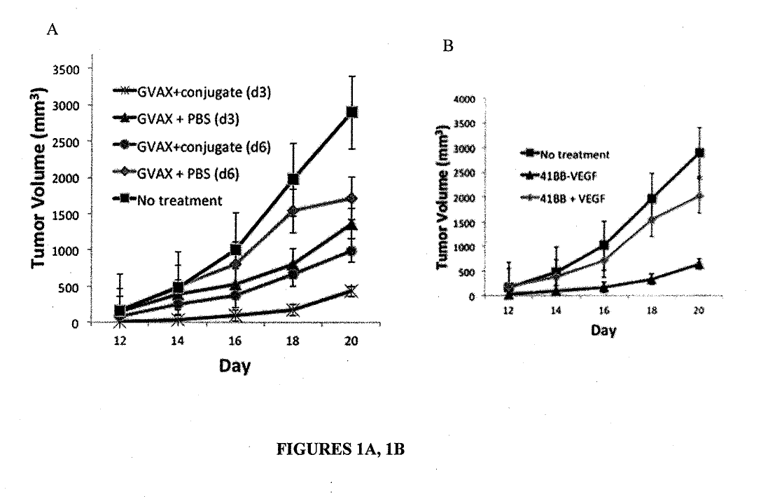

FIGS. 1A and 1B show the results from experiments whereby mice were implanted with tumor, melanoma B16, and at day 3 or day 6 as indicated subjected to treatment which included either vaccination alone (GVAX) or vaccination with a bi-specific aptamer (conjugate) consisting of 4-1BB fused to VEGF. The conjugate enhanced tumor immunity. FIG. 2B (panel on the right) is a control w/o vaccination when treatment started at day 3 (with more material) which shows that the antitumor effect of the conjugate requires physical linkage, because 4-1BB and VEGF as a mixture had a minor effect by comparison.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A composition comprising: a multi specific agent having specificities for at least at two molecules, wherein a first domain is specific for a secreted cellular molecule and a second domain is specific for an immune cell modulatory molecule.

2. The composition of claim 1, wherein the agent comprises: aptamers, antibodies, antibody fragments, oligonucleotides, mimetics, peptides or small molecular weight (MW) compounds which bind to secreted products.

3. The composition of claim 2, wherein the small molecular weight compounds bind to secreted cellular molecules.

4. The composition of claim 3, wherein the small molecular weight compounds bind to metalloproteases.

5. The composition of claim 1, wherein the multi-specific binding agent is specific for at least two immune cell modulatory molecule and at least one secreted cellular molecule.

6. The composition of claim 1, wherein the multi-specific binding agent is specific for at least two secreted cellular molecules and at least one immune cell modulatory molecule.

7. The composition of claim 1, wherein the immune modulatory molecule is an immune stimulatory molecule.

8. The composition of claim 1, wherein the immune modulatory molecule is an immune inhibitory molecule.

9. The composition of claim 1, wherein the multi-specific binding ligand is a bi-specific aptamer.

10. The composition of claim 1, wherein the secreted cellular molecule comprises molecules secreted by cells in tumor stroma comprising: CCL21, sialoproteins, cytokines, growth factors, tumor antigens, tumor associated antigens, peptides, or combinations thereof.

11. The composition of claim 10, wherein growth factors, cytokines and angiogenic factors comprise: Vascular endothelial growth factor (VEGF), tumor necrosis factors (TNF) transforming growth factors (TGF), colony stimulating factors (CSF), Fibroblast growth factors (FGF), epidermal growth factor (EGF), platelet-derived growth factor (PDGF), interferons (IFN), interleukins, endostatins, osteopontin (bone sialoprotein (BSP)), or fragments thereof.

12. The composition of claim 1, wherein the secreted cellular molecule comprises secreted inflammatory molecules.

13. The composition of claim 1, wherein the secreted cellular molecule comprises molecules secreted by cells in tissues or organs subjected to an autoimmune reaction.

14. The composition of claim 7, wherein an immune cell stimulatory molecule comprises: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Ra), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2, CD150 (SLAM), CD152 (CTLA-4), CD154, (CD40L), CD178 (FasL), CD209 (DC-SIGN), CD270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles or fragments thereof.

15. The composition of claim 8, wherein an immune cell inhibitory molecule comprises: CTLA-4 (CD152), PD-1, or BTLA.

16. The composition of claim 1, optionally comprising one or more cargo moieties comprising: a chemotherapeutic agent, toxin, radioactive agent, enzyme, small molecule, organic compound, inorganic compound, or combinations thereof.

17. A method of treating cancer comprising administering to a patient a therapeutically effective amount of a composition of claim 1.

18. A method of treating autoimmune related diseases or disorders comprising administering to a patient a therapeutically effective amount of a composition of claim 1.
